(19) United States
(12) Patent Application Publication
(10) Pub. No.: US 2022/0072078 A1
(43) Pub. Date: Mar. 10, 2022

(54) COMPOSITIONS AND METHODS FOR TREATING BURNS, WOUNDS, AND SKIN DISORDERS

(71) Applicant: Katherine Echo KIM, Westport, CT (US)

(72) Inventor: Katherine Echo KIM, Westport, CT (US)

(21) Appl. No.: 17/417,703

(22) PCT Filed: Dec. 19, 2019
(86) PCT No.: PCT/US2019/067432

§ 371 Date: Jun. 23, 2021

Related U.S. Application Data
(60) Provisional application No. 62/784,909, filed on Dec. 26, 2018.

(51) Int. Cl. A61K 36/81; A61K 31/718; A61P 17/02; A61K 33/44

(52) U.S. Cl. CPC A61K 36/81; A61K 33/44; A61P 17/02; A61K 31/718

(57) ABSTRACT

The present invention provides compositions and methods for treating burns, wounds, and skin disorders. The compositions are based on the blackened, milled powder, i.e. ash, derived from processing the tuberous portion of a plant, such as the potato, at a high temperature.

### CROSS-REFERENCE TO RELATED APPLICATIONS

This application is a national phase entry under 35 U.S.C. § 371 of International Application PCT/2019/067432, filed Dec. 19, 2019, which claims the benefit under 35 U.S.C. § 119(e) of U.S. Provisional Application No. 62/784,909 filed Dec. 26, 2018, all of which are herein incorporated by reference in their entirety.

### FIELD OF THE INVENTION

The present invention provides compositions and methods for treating burns, wounds, and skin disorders. The compositions are based on the charred powder, i.e. ash, derived from processing the tuberous portion of a plant, such as the potato, at a high temperature.

### BACKGROUND

The search continues for ever more effective remedies to treat skin burns and wounds. While current burn treatments often involve using cold therapeutics, cold water, cool compresses, and assorted remedies, the novelty of this present invention relies on the therapeutic principle of “like treating like” applying a burned composition to treat dermal burns. For example, the concept of “like cures like” may have been suggested by Hippocrates around 400 BC, when he prescribed a small dose of mandrake root to treat mania, knowing it produces mania in much larger doses. (See, Hemenway, Henry Bixby (1894). “Modern Homeopathy and Medical Science”. JAMA: The Journal of the American Medical Association. XXII (11): 367. doi:10.1001/jama.1894.02420900001001.) Similarly, in the 16th century, Paracelsus wrote “similia similibus curantur” (similar to the subjunctive form later used by Hahnemann), often translated as “what makes a man ill also cures him”. (See, Paracelsus (German-Swiss physician)”. Britannica Online Encyclopedia. Encyclopdia Britannica.)

There is a long history of “like treating like” in medicine, ecology, cooking, and others. It is well known that one can quickly put out a fire by throwing ash on it. Essentially, the present invention utilizes a charred plant-based material to treat burns, skin wounds, and other skin disorders.

In ecology, one of the oldest and most effective methods of protecting wood from fire is fire itself. The process of applying heat and fire to the exterior of a wood product for a short period of time changes both the cellular structure and thermodynamic conductivity of the wood. When wood is burned, the softer, more reactive cellulose vaporizes and is burned off, while the harder lignin takes a longer time to burn. Additionally, the outer layer of charred wood acts as an insulator. For example, the Japanese construction technique of charring wood surfaces, is known as shou-sugi-ban. This chemical-free preservation technique is valued because it envelops wood in a layer of carbon that is highly resistant to invasion from mold, insects, water and even more fire. (See, Burning wood preserves it “Shou Sugi Ban: The Traditional Art of Charred Cedar” https://permaculturenews.org/2016/12/05/shou-sugi-ban-preserve-wood-using-fire/.) Also, burned logs are left on forest floors in permaculture. Controlled fires also help forests germinate. Wildfires are left to naturally heal without thinning the forests.

In the area of food making and preservation, charred vegetable ash is used in cheesemaking to protect cheese from contamination and insect invasion. Vegetable ash is believed to neutralize the surface pH of cheese. (See, https://food.good.is/features/cheese-vegetable-ash.) It is believed that the ash preserves the cheese by discouraging microbes and mold spores. It has also been observed that the ash tends to dry off the surface of the cheese as well. The ash applied to cheese once came directly from a fire but is now mainly made from salt and vegetable ash (vegetables that are dried and turned into ash). The ash is sterile, odorless and flavorless. It is a finely powdered, food-grade, sterile component actually revered by the medical world for its ability to control and absorb toxins. (See, https://www.thespruceeats.com/why-is-ash-used-in-cheesemaking-591201.)

Burns, wounds, and skin disorders represent a significant health problem worldwide. Although a variety of treatments and medications have been available since ancient times and are continually being developed, there are often disadvantages with these treatments and medications including painful debridement methods, higher risk of infection, and scarring. It would therefore be highly useful to have new treatments and medications that are relatively easy to prepare and use and that are safe and effective.

To further clarify, there are three primary types of burns: first-, second-, and third-degree. Each degree is based on the severity of damage to the skin, with first-degree being the most minor and third-degree being the most severe. Damage includes:
first-degree burns: red, non-blistered skin
second-degree burns: blisters and some thickening of the skin
third-degree burns: widespread thickness with a white, leathery appearance
there are also fourth-degree burns. This type of burn includes all of the symptoms of a third-degree burn and also extends beyond the skin into tendons and bones. (https://www.healthline.com/health/burns#burn-levels)

It is therefore seen that there is a need to provide compositions and methods for treating, burns, wounds, and skin disorders. The present invention addresses the shortcomings of current compositions and methodologies.

### SUMMARY OF THE INVENTION

The present invention provides compositions and methods for treating a range of burns, wounds, and skin disorders based on the burned, blackened, milled powder derived from the tuber of a tuberous plant.

### BRIEF DESCRIPTION OF THE DRAWINGS

FIG. 1 shows a flow chart for preparing a composition of the present invention.

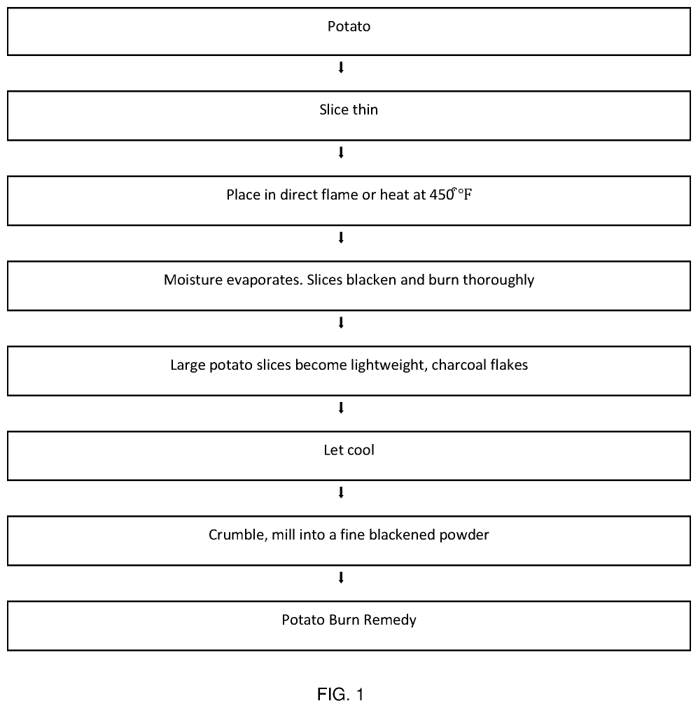

### DETAILED DESCRIPTION OF THE INVENTION

The present invention relates to a composition comprising as an active ingredient the powder, i.e. the ash, derived from heating to a high temperature, or burning, the tuberous portion of a tuberous plant.

In other embodiments, the present invention relates to a composition wherein the tuberous plant can be selected from one or more of the following geniuses and/or species: Solanum tuberosum, Ipomoea batatas, Manihot esculenta, and Dioscorea, such as Dioscorea rotundata, Dioscorea cayenensis, Dioscorea elate, Dioscorea polystachya, Dioscorea bulbifera, Dioscorea esculenta, Dioscorea dumetorum, and Dioscorea trifida.

In other embodiments, the present invention relates to a composition wherein the tuberous plant is selected from Solanum tuberosum.

In other embodiments, the present invention relates to a composition comprising a starch selected from the group consisting of amylose, amylopectin, and combinations thereof. In other embodiments, the present invention relates to a having a water content of less than about 20% by weight, or less than about 15% by weight, or less than about 10% by weight, or less than about 5% by weight, or less than about 2% by weight, or less than about 1% by weight, or less than about 0.5% by weight, or less than about 0.1% by weight, based on the total weight of the composition, and wherein the residual water can be at 0.01% or less.

In other embodiments, the present invention relates to a composition having an average particle size of about 0.1 to about 100,000 micrometers, or about 1 to about 10,000 micrometers, or about 10 to about 1000 micrometers.

In other embodiments, the present invention relates to a composition of the present invention formulated as a topical composition.

In other embodiments, the present invention relates to a method for preparing the composition of the present invention comprising the steps of:

(a) thinly slicing the tuberous portion of a tuberous plant,

(b) heating the slices to a temperature of about 450° F. or placing the slices in a direct flame until dried and charred,

(c) cooling the resulting burned material to about room temperature,

(d) milling the resulting material to produce a fine blackened powder.

In other embodiments, the present invention relates to a method wherein step (b) is performed until there is no further change in weight of the sample, compared to prior to heating.

In other embodiments, the present invention relates to a method for treating a condition selected from a skin burn, a skin wound, or a skin disorder comprising

(a) applying a composition according to claim 1 to the skin of an afflicted subject,

(b) letting the composition remain in contact with the skin of the afflicted subject,

(c) allowing the composition to absorb pus-like discharge, harden into a scab, then fall or slough off from the skin of the afflicted subject.

In other embodiments, the present invention relates to a method of comprising the further step of (d) covering the treated skin with a dressing.

In other embodiments, the present invention relates to a method comprising repeating steps (a), (b), and (c) one or more additional times until the desired therapeutic effect is achieved, or comprising repeating steps (a), (b), (c), and (d) one or more additional times until the desired therapeutic effect is achieved.

In other embodiments, the present invention relates to a method for treating a condition selected from a skin burn, a skin wound, or a skin disorder comprising

(a1) cleaning debris from the burn, wound, or disorder,

(b1) applying a composition according to claim 1 to the skin of an afflicted subject,

(c1) allowing the composition remain in contact with the skin of the afflicted subject,

(d1) allowing the composition to absorb and retain as much pus-like discharge from the burn, wound, or disorder

(e1) allowing the composition and all exudate to form into a hardened scab

(f1) allowing the composition to keep the wound moist underneath the scab yet cover all entry points to the wound, creating a natural protective barrier,

(g1) allowing the composition to remain adhered to the skin while subdermal layers regenerate/heal,

(h1) allowing the composition to naturally fall or slough off from the skin of the afflicted subject without debridement or other interference to prevent the chance of scarring.

In other embodiments, the present invention relates to a method comprising the further step of applying the composition as a cream, spray, gel ointment, wound dressing to allow greater ease of application and adherence.

These and other aspects of the present invention will become apparent from the disclosure herein.

### Compositions of the Present Invention

There are many types of tuberous plants. Tubers are the thickened underground portion of the stem or rhizome of certain plants. A common tuber is the potato. Botanically, potatoes and sweet potatoes are completely unrelated. Potatoes (Solanum tuberosum) are in the Solanaceae family, related to tomatoes, peppers, and eggplant along with deadly nightshade. Sweet potatoes relate to a vine species (Ipomoea batatas) and is a dicotyledonous plant in the bindweed or morning glory family (Convolvulaceae). In the United States and Canada, sweet potatoes are often (confusingly) referred to as “yams.” But sweet potatoes are not related to yams.

There are about 5,000 potatoes varieties worldwide. Three thousand of them are found in the Andes alone, mainly Peru, Bolivia, Ecuador, Chile, Columbia. They belong to eight to nine species depending on taxonomic schools. Apart from 5,000 cultivated species varieties, there are about 200 wild varieties and subspecies which can be cross-bred. (https://en.wikipedia.org/wiki.Potato). A potato is 80% water and 20% solids.

The compositions of the present invention are useful for treating skin conditions such as burns and wounds. These compositions are based on a charred powder derived from the tuberous portion of tuberous plants. Tubers from tuberous plants useful herein include: potatoes (white) such as Solanum tuberosum, sweet potatoes such as Ipomoea batatas, cassava roots such as Manihot esculenta, yams such as those of the genus Dioscorea, (for example, Dioscorea rotundata, Dioscorea. cayenensis, Dioscorea alata, Dioscorea polystachya, Dioscorea bulbifera, Dioscorea esculenta, Dioscorea dumetorum, and Dioscorea trifida), lotus such as Nelumbo nucifera, taro such as Colocasia esculenta, ginseng such as Panax, radishes such as those of the genus Raphanus, ginger such as Zingiber originalis, and the like. White potatoes, particularly the waxy white potato varieties, produce two main kinds of potato starch, namely amylose and amylopectin. It is believed that the charred powder derived from these starches are useful to preparing the compositions of the present invention.

### Methods of Making the Compositions

The compositions of the present invention are prepared using convention preparation techniques and equipment. An embodiment of the method of the present invention comprises the following steps:

(a) thinly slicing the desired amount of the tuberous portion of a tuberous plant,

(b) heating the slices to a temperature of about 450° F. (higher or lower temperatures can be used depending on the quantity of the tuberous plant and the expected preparation time) or placing the slices in a direct flame until dried and charred,

(c) cooling the resulting charred material to about room temperature,

(d) milling the resulting charred material to produce a blackened powder.

Generally, step (b) is performed until there is no further change in weight of the sample, compared to prior to heating. The objective is to obtain a dehydrated material that has little to no residual water. The compositions can have a water content of less than about 20% by weight, or less than about 15% by weight, or less than about 10% by weight, or less than about 5% by weight, or less than about 2% by weight, or less than about 1% by weight, or less than about 0.5% by weight, or less than about 0.1% by weight, based on the total weight of the composition. There is no specific lower range for residual water, but generally a lower water level content should be 0.01% or less.

The objective is to obtain lightweight charcoal flakes that readily crumble and can be crushed or milled into a fine blackened powder. Particle sizes can range from a composition having an average particle size of about 0.1 to about 100,000 micrometers, or about 1 to about 10,000 micrometers, or about 10 to about 1000 micrometers.

The resulting powder can be used as is, or can further be formulated in to an appropriate vehicle for topical delivery. Examples of such vehicles include oils, solutions, suspensions, emulsions, and burn wound care dressings such as adhesive bandages.

### Methods of Treating Burns, Wounds and Other Skin Disorders

The compositions of the present invention are useful for treating various skin injuries, particularly in mammals such as humans. These skin injuries include burns and wounds. The compositions of the present invention can be applied directly to the burn or wound to cover it. The composition is then allowed to air dry and form into a protective layering or scab. More composition is to be applied to absorb pus-like exudate until all discharge stops. The composition is generally allowed to remain on the skin or the subject until it sloughs or falls off on its own and the desired treatment effect is achieved.

### EXAMPLES

The following examples further describe and demonstrate embodiments within the scope of the present invention. The Examples are given solely for purpose of illustration and are not to be construed as limitations of the present invention, as many variations thereof are possible without departing from the spirit and scope of the invention.

### Example 1 White Potato Composition and Method

A desired quantity of raw potato is cut into thin slices. The slices are placed directly in to a flame or alternatively placed in a heat proof vessel and heated over a heat source. The potato slices are heated to evaporate any moisture and to blacken or burn the slices. The heat or flame source is applied until lightweight blackened dry charcoal-type flakes are obtained. The resulting burnt flakes are cooled to room temperature. The cooled flakes are milled into a fine blackened powder. The resulting composition is applied directly to a skin burn or wound and left to absorb any pus-like discharge, dry, harden into a scab, crust over, and slough off on its own. The composition is useful for treating skin burns or wounds. The application of the composition can be repeated to treat the skin burn or wound as necessary. In alternative embodiments the composition and method can be made and performed by replacing the white potato with the desired quantity of other tubers.

### REFERENCES

U.S Patent Application No. US20130337090, Plant Extracts for Treating Burns and Chronic Wounds, to Shirli Doron, published Dec. 13, 2013.
U.S. Pat. No. 8,784,903, Medicinal Plant Extract Using Processing of Herbal Medicine and Composition of Skin External Application Comprising the Same, to Jun Seong Park, et al., issued Jul. 22, 2014.
Chinese Patent Publication No. CN107412343A, A method of treating eczema and burns topical coating agent and preparation method thereof, to Ming Kang, published Dec. 1, 2017.
Chinese Patent Publication No. CN1122709A, Medicinal powder “Shulingsan” and its preparation containing sweet potato, to Xijin Chai, published May 22, 1996.
Daniele Hermes et al., In vivo wound healing and antiulcer properties of white sweet potato (Ipomoea batatas), J. Adv. Research 2013, published Jul. 6, 2012.
Chinese Patent Publication No. CN101336947A, Deer Hemostatic Powder, to Jun Chen, published Jan. 7, 2009.
Chinese Patent Publication No. CN105363055A, Herbaceous incense and preparation method thereof, to Xiaoli Yang, published Mar. 2, 2016.
Ereth et al., Microporous polysaccharide hemospheres provides effective topical hemostasis in a human modified bleeding time incision model, 2002.
U.S. Pat. No. 9,132,114, Composition and Method to Treat Wounds, to David Dixon, issued Sep. 15, 2015.
U.S Patent Application No. US20060258560, Dry Tissue Sealent Compositions, to Chunlin Yang et al., published Nov. 16, 2006.
U.S. Pat. No. 6,046,160, Composition and Method for Enhancing Wound Healing, to Obi-Tabot et al., issued Apr. 4, 2000.
Fernando Torres et al., Starch Torres 2013, Starchbased biomaterials for wound dressing applications, published May 3, 2013.
U.S Patent Application No. US20090035289, Dry Platelet Composition, to Christopher Wagner et al., published Feb. 5, 2009.
Patent Biotech 2015 Abstract, Products with natural components to heal dermal burns: a patent review, to Santana de Melo Costa et al., published Dec. 1, 2015.
Toshio Suzuki et al., Bio Pharma Bulletin, Application of Sweet Potato Fiber to Skin Wound in Rat, published Jul. 1, 1996.
Chinese Patent No. CN1293897C, Burn- and Scald-treating powder, to Jinfeng Chen, issued Jan. 10, 2007.
Hemenway, Henry Bixby (1894). “Modern Homeopathy and Medical Science”. JAMA: The Journal of the American Medical Association. XXII (11): 367. doi:10.1001/jama.1894.02420900001001.
Burning wood preserves it “Shou Sugi Ban: The Traditional Art of Charred Cedar” https://permaculturenews.org/2016/12/05/shou-sugi-ban-preserve-wood-using-fire/.

### INCORPORATION BY REFERENCE

The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps or components, it is also contemplated that the methods and systems consist essentially of, or consist of, the recited steps or components. Furthermore, the order of steps or order for performing certain actions is immaterial as long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

Furthermore, it should be recognized that in certain instances a composition can be described as composed of the components prior to mixing, because upon mixing certain components can further react or be transformed into additional materials.

All percentages and ratios used herein, unless otherwise indicated, are by weight.

1. A composition comprising as an active ingredient the charred powder derived from the tuberous portion of a tuberous plant.

2. A composition according to claim 1 wherein the tuberous plant is selected from one or more of the following geniuses and/or species: Solanum tuberosum, Ipomoea batatas, Manihot esculenta, and Dioscorea, such as Dioscorea rotundata, Dioscorea. cayenensis, Dioscorea alata, Dioscorea polystachya, Dioscorea bulbifera, Dioscorea esculenta, Dioscorea dumetorum, and Dioscorea trifida.

3. A composition according to claim 1 wherein the tuberous plant is selected from Solanum tuberosum.

4. A composition according to claim 1 comprising a starch selected from the group consisting of amylose, amylopectin, and combinations thereof.

5. A composition according to claim 1 having a water content of less than about 20% by weight, or less than about 15% by weight, or less than about 10% by weight, or less than about 5% by weight, or less than about 2% by weight, or less than about 1% by weight, or less than about 0.5% by weight, or less than about 0.1% by weight, based on the total weight of the composition, and wherein the residual water can be at 0.01% or less.

6. A composition according to claim 1 having an average particle size of about 0.1 to about 100,000 micrometers, or about 1 to about 10,000 micrometers, or about 10 to about 1000 micrometers.

7. A composition of claim 1 formulated as a topical composition.

8. A method for preparing the composition of claim 1 comprising the steps of:
(a) thinly slicing the tuberous portion of a tuberous plant,
(b) heating the slices to a temperature of about 450° F. or placing the slices in a direct flame until dried and charred,
(c) cooling the resulting charred material to about room temperature,
(d) milling the resulting charred material to produce a blackened powder.

9. A method according to claim 8 wherein step (b) is performed until there is no further change in weight of the sample, compared to prior to heating.

10. A method for treating a condition selected from a skin burn, a skin wound, or a skin disorder comprising
(a) applying a composition according to claim 1 to the skin of an afflicted subject,
(b) letting the composition remain in contact with the skin of the afflicted subject,
(c) allowing the composition to absorb pus-like discharge, harden into a scab, then fall or slough off from the skin of the afflicted subject.

11. A method according to claim 10 comprising the further step (d) of covering the treated skin with a dressing.

12. A method according to claim 10 comprising repeating steps (a), (b), and (c) one or more additional times until the desired therapeutic effect is achieved.

13. A method for treating a condition selected from a skin burn, a skin wound, or a skin disorder comprising
(a) cleaning debris from the burn, wound, or disorder,
(b) applying a composition according to claim 1 to the skin of an afflicted subject,
(c) allowing the composition remain in contact with the skin of the afflicted subject,
(d) allowing the composition to absorb and retain as much pus-like discharge from the burn, wound, or disorder
(e) allowing the composition and all exudate to form into a hardened scab
(f) allowing the composition to keep the wound moist underneath the scab yet cover all entry points to the wound, creating a natural protective barrier,
(g) allowing the composition to remain adhered to the skin while subdermal layers regenerate/heal,
(h) allowing the composition to naturally fall or slough off from the skin of the afflicted subject without debridement or other interference to prevent the chance of scarring.

14. A method comprising the applying the composition of claim 1 as a cream, spray, gel ointment, wound dressing to allow greater ease of application and adherence.

15. A topical skin treatment composition comprising as an active ingredient the charred powder derived from the tuberous portion of Solanum tuberosum, wherein the powder has a particle size from about 0.1 micrometers to about 1000 micrometers and the composition has a water content of less than about 20% by weight based on the total weight of the composition.

16. The composition according to claim 15 wherein the charred powder is obtained by heating the tuberous portion of Solanum tuberosum to a temperature of about 450° F. until charred and no further change in weight of the material is observed compared to prior to heating.

17. The composition according to claim 15 wherein the charred powder is obtained by burning the tuberous portion of Solanum tuberosum until charred and no further change in weight of the material is observed compared to prior to heating.

18. The composition according to claim 15 further formulated into a vehicle selected from the group consisting of oils, solutions, suspensions, emulsions.

19. The composition according to claim 15 further formulated into a burn wound care dressing.

20. The composition according to claim 19 wherein the burn wound care dressing is an adhesive bandage.
